(19) United States
(12) Patent Application Publication
(10) Pub. No.: US 2006/0247423 A1
(43) Pub. Date: Nov. 2, 2006

(54) Hemoglobin conjugate and the preparation method and its use

(76) Inventors: Zhiguo Su, Beijing (CN); Lu Xiuling, Beijing (CN); Zheng Chunyang, Bejing (CN); Xu Yuhong, Beijing (CN)

Correspondence Address: VENABLE LLP, P.O. BOX 34385, WASHINGTON, DC, 20045-9998, US

(21) Appl. No.: 10/551,931

(22) PCT Filed: Oct. 5, 2005
(86) PCT No.: PCT/CN04/00091

§ 371 Date: Oct. 5, 2005

(30) Foreign Application Priority Data
Apr. 9, 2003 (CN) 03109622.0

(51) Int. Cl. A61K 38/42; C07K 14/805

(52) U.S. Cl. 530/363; 530/385; 514/6

(57) ABSTRACT

The present invention relates to conjugates of hemoglobin and human serum albumin and the preparation process thereof. The conjugates have molecular weight in the range of 100-300 kD, comprising 1-3 intermolecularly or intramolecularly cross-linked hemoglobin molecules and 1-3 human serum albumin molecules. The conjugates are obtained by the following steps: preparing stroma-free hemoglobin, then coupling hemoglobin to human serum albumin and purifying the products using anion exchange chromatography. The hemoglobin and human serum albumin conjugates have characteristics suitable for being used as blood substitutes.

### FIELD OF THE INVENTION

The invention relates to hemoglobin-based blood substitutes and the preparation processes thereof. More specifically, it relates to conjugates of hemoglobin and human serum albumin, the preparation method and the use as blood substitutes.

### BACKGROUND OF THE INVENTION

Blood transfusion is an important medical treatment of clinic surgery and rescue operation in emergent disasters and military conflicts. However, transfusion of a patient with donated blood has a number of disadvantages. To infuse blood, blood typing is required, which takes at least several tens of minutes to decide the correct blood type of the patient before the transfusion can be performed. Furthermore, there may be a shortage of blood supply due to lack of donors, lack of special equipment for storage and transportation of blood. And the increasing risk of virus disease infection, such as infection by hepatitis virus and HIV, really endangers the blood transfusion. In recently year, the demand of blood is increasing, while at the same we are short of safety blood supply. Thus, researchers worldwide have paid much attention to the development of human blood substitute.

Human blood substitute also know as human red cell substitutes, are artificially agents that have functions of delivering oxygen, maintaining colloid osmotic pressure, and expanding the blood vessels. Ideal blood substitutes have good properties of oxygen carrying ability, biocompatibility, safety and stability, which are similar to red blood cells. Blood substitutes could be widely used for: red blood cell replacement in surgical settings; emergency resuscitation of traumatic blood loss; shorter term red blood cell replacement in other conditions; other medical applications requiring oxygen delivery such as organ preservation, balloon angioplasties and thrombosis. Recently it has been developed as oxygen therapeutic, especially for curing tumor.

Two types of substitutes have recently been studied, chemosynthetic fluorocarbon emulsions and hemoglobin (Hb) solutions. Perfluorochemical-based compositions dissolve oxygen as opposed to binding it as a ligand. Though the perfluorocarbon emulsions are inexpensive to manufacture, they do not carry sufficient oxygen at clinically tolerated doses to be effective. The type of red blood cell can be changed but such transformed red blood cells still meet the problems of short term storage and inconvenient transportation. Hemoglobin comes from red blood cell, having good ability to carry oxygen and keep osmotic pressure, and could be more compatible compared to synthetic perfluorocarbon. Therefore, hemoglobin-base blood substitutes are the focus of the research, including modified hemoglobin, hemoglobin vesicles and encapsulated hemoglobin (Winslow R M. Hemoglobin-based red cell substitute. The John Hopkins University Press, Baltimore and London. 1992: 39).

In vivo stroma-free hemoglobin (SFHb) readily dissociates into alpha-beta dimers, and even further under some conditions to alpha subunit monomers and beta subunit monomers. The dimers and monomers have too low a molecular weight for retention in the circulatory system of the body, and are filtered by the kidneys for excretion with the urine. This results in an unacceptably short half life and significant nephrotoxicity. Moreover, free from red blood cells, hemoglobin may have the problems of high oxygen affinity and high colloid osmotic pressure. To solve these problems, a number of chemical modifications have been introduced into stroma-free hemoglobin in attempts to render the hemoglobin more stable, prolong the vascular retention of SFHb and reduce its renal toxicity. Hemoglobin comprises a tetramer of four subunits. Intramolecular cross-linking of Hb stabilized the structure. Increasing the molecular size could prevent modified Hb from being filtered by the kidneys and so extend the circulatory duration of Hb. However, not all modification decreased the immunity of Hb. Therefore, not only renal toxicity should be avoided by hemoglobin modification, but the bioactivity of Hb should be well kept and less immunity appearing. Typical modified Hb includes Polymerized intramolecularly cross-linked Hb (Rausch C W, Gawryl M S, Light W R et al. Stable polymerized hemoglobin blood-substitute. EP1093720. Apr. 25, 2001), modified Hb with macromolecules, such as polyethylene glycol (PEG) (Davis F, Nho K, Zalipsky S Chemically modified hemoglobin as an effective, stable non-immunogenic red blood cell substitute. U.S. Pat. No. 5,234,903. Aug. 10, 1993) or polysaccharide (Adamson G J. Hemoglobin-polysaccharide conjugates. U.S. Pat. No. 6,500,930. Nov. 31, 2002).

The blood substitutes developed overcome most problems of red blood cells, while none of the substitutes has all advantages of red blood cells. Currently, the research of blood substitutes is still facing many difficulties, especially the challenge of safety and efficacy. Some products were found vexing side effects such as vasoconstration and increasing blood pressure in clinical trials, which limit the application of blood substitutes, especially on old people. Red blood cell is far more sophisticated, containing a cytoskeleton and enzyme and transport systems that ensure hemoglobin flexibility for its 120-day lifespan, while the longest duration time of blood substitutes is less than seventy hours.

Serum albumin (Molecular weigh 67,000) is the largest fraction of plasma proteins that performs functions such as balancing osmotic pressure, carrying nutrient substances (fatty acid, amino acid, steroid, metal ions and drugs, etc), releasing toxicity by excluding toxic substrates. Human serum albumin (HSA) has been widely used as volume expander and complement in clinical surgery.

### DETAILED DESCRIPTION OF THE INVENTION

The present invention is directed to hemoglobin and human serum albumin conjugates (Hb-HSA conjugates). The conjugates are capable of delivering oxygen to tissues, keeping the osmotic pressure and carrying nutritious, performing more blood functions.

The present invention is also directed to the preparation method for the Hb-HSA conjugates.

In a preferred embodiment, the conjugates have molecular weight of between 100-300 KD. The conjugates comprise 1-3 hemoglobin molecules, preferably 1-2 hemoglobin molecules, and 1-3 human serum albumin molecules, preferably one HSA molecule. Hemoglobin of the conjugates is intramolecularly cross-linked or non-intramolecularly cross-linked, preferably intramolecularly cross-linked. Human or non-human hemoglobin may be used according to the invention. In a preferred embodiment, non-human hemoglobin from any appropriate animal source, including, but not limited to, bovine, ovine, equine, canine and porcine hemoglobin, may be used according to the invention.

In preferred embodiments of the invention, the procedure of the conjugates preparation including stroma-free hemoglobin preparation, hemoglobin and HSA coupling and the purification of Hb-HSA conjugates.

The preparation of stoma-free hemoglobin can use the convention means in techniques to produce stoma-free hemoglobin with electrophoretic or chromatographic purity.

In particular embodiments, the invention provides stroma-free hemoglobin preparation methods comprising membrane filtration and ion exchange chromatography. Highly purified hemoglobin with one band in SDS-PAGE and one peak in HPLC can be prepared by the integrated preparation method. Hypotonic salt solutions may be employed to swell the membrane of red blood cells and allow the hemoglobin to leak out gradually, while keeping the cell membrane intact (Doczi J. Injectable stroma free hemoglobin and its method of manufacture. 1976, U.S. Pat. No. 3,991,181). The hemolysate is processed through microfiltration membranes with mean pore size from 0.22 μm to 0.65 μm, preferably 0.45 μm, followed by ultrafiltration with molecular weight cut-off (MWCO) from 10 kD to 30 kD, preferably 30 kD to remove the ghost and cellular debris. The pretreated hemoglobin solution is further purified by flow through mode of anion exchange chromatography. In preferred embodiments of the invention, DEAE Sepharose Fast Flow QMA Spherosil LS, Q Sepharose Big Beads (Amersham phamacia, Sweden) may be used as chromatography media, and phosphate buffer solution (PBS) with a concentration of 10-100 mM, pH 6.6-8.5, and preferably 20-50 mM, pH 7.0-7.8. Adding polyethylene glycol (PEG) as an escort in ion exchange chromatography is preferred to improve the purity and recovery. PEG400-4000 may be applied with a concentration of 0.25-10%, and preferably 0.5-5%. The recovery of the chromatography in the present invention is more than 95%. All operations are conducted at 4-10° C. The prepared pure hemoglobin is used to couple with HSA.

In the invention, the cross-linking reagents to couple Hb and HSA may be all the bifunctional cross-linker that can react with amino groups, sulfhydryls and hydroxyls. Preferably, homo- or hetero-bifunctional cross-linker with aldehyde groups, N-hydroxysuccinimide ester groups (NHS), epoxide groups, maleimide groups and imido-ester groups may be used.

In preferred embodiments of the invention, the coupling methods for Hb and HSA may be one- or two-step coupling in solution, or reacting as the protein is absorbed on solid media.

One-Step Coupling:

Coupling reaction may be carried out in phosphate, HEPES, boric acid, borax-sodium hydroxide or sodium carbonate buffer solution with pH6-12, and preferably pH7.5-9.5. The concentration of Hemoglobin and HSA may be 1-150 mg/mL, and preferably 10-100 mg/mL. The molar ratio of cross-linker and protein may be 3:1-600:1, and preferably 10:1-200:1. The reaction may be performed at 4-55° C. for 0.1-48 h, and preferably at 25-37° C. for 0.5-10 h.

Two-Step Coupling:

Coupling reaction may be carried out in phosphate, HEPES, boric acid, borax-sodium hydroxide or sodium carbonate buffer solution, pH6-12, and preferably pH7.0-9.5. Hemoglobin or HSA may first react with cross-linker with a concentration of 1-150 mg/mL, and preferably 5-60 mg/mL. The molar ratio of cross-linker and protein may be 3:1-600:1, and preferably 10:1-500:1. The reaction may be performed at 4-55° C. for 0.1-48 h, and preferably at 25-37° C. for 0.5-10 h. Unreacted cross-linker may be removed by column of Sephadex G25 or dialysis, and the pH of the solution can be the same or changed to 6-12, and preferably 7.5-9.5 at the same time. Then another protein may be added to react for 1-48 h.

Solid Phase Coupling:

In preferred embodiments of the invention, anion or cation exchange media may be applied as solid phase, and preferably using DEAE Sepharose Fast Flow, Q Sepharose Big Beads, Q Sepharose Fast Flow, SP Sepharose Fast Flow or CM Sepharose Fast Flow (Amersham phamacia, Sweden). Hemoglobin or HSA solution with concentration of 0.5-5 mg/mL is mixed with the solid media that have been equilibrated by 50 mM PBS or HEPES buffer with pH 4.0-8.5, and preferably 4.5-7.5, followed by thorough washing till no protein was present in the eluate. Cross-linker solution is added at molar ratio of 30:1-600:1 with protein, and preferably 10:1-500:1. The column may be closed for 0.1-12 h, and preferably 0.5-10 h, and then washed extensively with the above equilibration buffer to remove excessive cross-linker, followed by elution with 0.5 M NaCl in the equilibration buffer to collect the active protein by cross-linker. The fractions may be passed through a pre-packed Sephadex G-25 gel filtration column to desalt, followed by concentration with pressure filtration, obtaining 1-5 mg/mL active protein, and the pH of the solution can be the same or changed to 6-12, and preferably 7.5-9.5 at the same time. Another protein may be added to react for 1-24 h, and then hemoglobin and HSA conjugates with molar ratio 1:1 will be obtained.

In preferred embodiments of the invention, the Hb-HSA conjugates may be purified by one or two or three of the purification procedures selected from ion exchange chromatography, ultrafiltration and gel filtration chromatography, to remove uncoupled components and the conjugates with Mw more than 300 kD and less than 100 kD.

In preferred embodiments of the invention, the pH value of purified Hb-HSA conjugates may be adjusted to 7.4, and 2,3-diphosphoglycerate (2,3-DPG) or pyridoxal-5′-phosphate may be added as covalence regulator for those hemoglobins whose ability of oxygen carrying can be effect by 2,3-DPG. The hemoglobin and human serum albumin conjugates have good characteristics for using as a blood substitute.

The Hb and HSA conjugates being used as blood substitutes have significant advantages. Hemoglobin is the main protein in red blood cell, which is responsible for oxygen delivering to tissues. Serum albumin is the most abundant protein in the plasma that performs functions such as balancing osmotic pressure, carrying nutrient substances, releasing toxicity by excluding toxic substrates, etc. The conjugates have stable functions of both proteins. The enlarged molecular weight and decreased pI for coupling with HSA (pI4.9) can extend the circulatory duration of Hb. The narrow distribution of moderate molecular weight is helpful for avoiding congregation of polyHbs with high molecular weight. Furthermore, less immunity is expected due to conjugation with human serum albumin. Therefore, the Hb-HSA conjugates may be better blood substitutes with characteristics closer to real blood than previous substitutes. The invention provides conjugates of HSA with hemoglobin from human, bovine, ovine, equine, canine and swine, to be used as blood substitutes.

### DESCRIPTION OF THE FIGURES

FIG. 1A is a scanning profile of gel electrophoresis (SDS-PAGE) for red blood cell lysate;

FIG. 1B is a scanning profile of SDS-PAGE for purified hemoglobin;

FIG. 2 is the size exclusion HPLC analysis curve of purified hemoglobin on TSK 3000^{SW}, detected at 280 nm;

FIG. 3 is a chromatogram illustrating the purification of the conjugates by ion exchange chromatography (IEC) with media of DEAE Sepharose Fast Flow;

FIG. 4 is gel filtration chromatography analysis curve of the conjugates on Superdex 200;
a described the protein peak pooled by IEC method, which was applied on gel filtration column;
b described the reaction mixture applied on gel filtration column;

FIG. 5 is characterization of the conjugates using SDS-PAGE;
Lane 1 is standard marker; lane 2 is the conjugates; lane 3 is native BSA, lane 4 is purified native hemoglobin;

FIG. 6 is the oxygen equilibrium curves of the conjugates;

FIG. 7 is size-exclusion HPLC analysis of Hb-HSA conjugates;

FIG. 8 is a diagram showing the effects of the long term survivals in the lethal hemorrhagic shock rat model with bleeding.

### THE PREFERRED EMBODIMENTS

### Example 1

### Preparation of Stroma-Free Bovine Hemoglobin

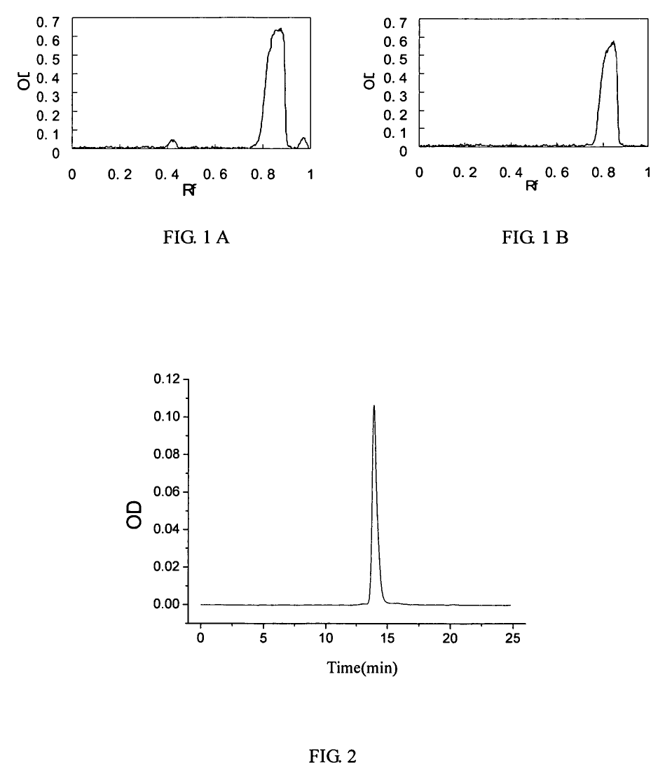

Bovine blood was collected, and clean packed cells were swelled and hemolyzed after being diluted with ice-cold 20 mM KH₂PO₄/Na₂HPO₄ (PBS) buffer solution containing 0.6% NaCl (pH7.4), and shaken for 1 h at 4° C. Then double RBC volume of PBS was pumped at the rate of 10% per minute, then continued to shake for 1 h and NaCl concentration adjusted to 0.9% to obtain the hemolysate. The hemolysate was processed through Millipore Pellicon membrane system to remove the ghost and cellular debris. Microfiltration membranes with mean pore size 0.22 μm was used, and the filtrate was then put through MWCO 10K ultrafiltration membrane to concentrate the hemoglobin and to remove low molecular weight impurities. The pretreated hemoglobin solution was further purified by flow-through mode of anion exchange chromatography with media of DEAE Sepharose Fast Flow (Amersham phamacia, Sweden), and 5% PEG400 was added as an escort. The column was equilibrated with 5 column volumes of 20 mM PBS containing 5% PEG400, pH7.4. Loading and rinsing buffers were the same as equilibration buffer. Fractions directly flowing through during loading were collected and appeared as a single band in SDS-PAGE, which was shown in FIG. 1. All operations were conducted at 10° C. The recovery in the chromatography was 97%. The activity of hemoglobin, in terms of P₅₀ and Hill coefficient, was measured by Hemox™ Analyzer (TCS Scientific Corp., PA), and P₅₀ and Hill coefficient were 25.6 mmHg and 2.41 respectively.

### Example 2

### Preparation of Stroma-Free Swine Hemoglobin

Porcine blood was collected, and clean packed cells were swelled and hemolyzed after being diluted with ice-cold 20 mmol/L KH₂PO₄/Na₂HPO₄(PBS) buffer solution containing 0.6% NaCl (pH7.4), and shaken for 1 h at 4° C. Then double RBC volume of PBS was pumped at the rate of 10% per minute, then continued to shake for 1 h and NaCl concentration adjusted to 0.9% to obtain the hemolysate. The hemolysate was processed through Millipore Pellicon membrane system to remove the ghost and cellular debris. Microfiltration membranes with mean pore size 0.45 μm was used, and the filtrate was then put through MWCO 30 KD ultrafiltration membrane to concentrate the hemoglobin and to remove low molecular weight impurities. The pretreated hemoglobin solution was further purified by flow-through mode of anion exchange chromatography with media of Q Sepharose Big Beads (Amersham phamacia, Sweden), and PEG was added as an escort. The column was equilibrated with 10 mM PBS containing 0.5% PEG4000, pH7.8. Loading and rinsing buffers were the same as equilibration buffer. Fractions directly flowing through during loading were collected and appeared as a single peak in size exclusion HPLC, which was given in FIG. 2. All operations were conducted at 4° C. The recovery in the chromatography was 95%.

### Example 3

### One-Step Coupling of Human Hemoglobin and HSA with 3-Maleimidobenzoic Acid Nhydroxysuccinimide Ester (MBS)

MBS may react with the thiol groups of HSA so that affect the coupling reaction. To block the thiol groups of HSA, 10 ml of 5 mg/mL HSA (HEPES buffer, pH 7.8) was alkylated with 0.2 ml 30 mM iodoacetamide for 20 min and then reacted to 0.5 ml 30 mM MBS for 30 min. Excess reagents were removed by passage through Sephadex G-25. Hemoglobin was deoxygenated under N₂ for 2 h. The resultant HSA then reacted with 10 ml of 20 mg/mL deoxygenated hemoglobin for 2 h, followed by addition of 0.2 ml 30 mM iodoacetamide to terminate further reactions. All reactions were carried out at room temperature.

### Example 4

### Two-Step Coupling of Ovine Hemoglobin and HSA with Ethylene Glycol Diglycidyl Ether (EGDE)

Coupling reaction was carried out in 50 mM HEPES buffer, pH7.5. Hemoglobin was first reacted with cross-linker with a concentration of 60 mg/Ml. The molar ratio of EGDE and Hb was 500:1. The reaction was performed at 37° C. for 10 h with volume of 10 ml. Unreacted cross-linker may be removed by column of Sephadex G25, and the buffer was changed to 50 mM borax-sodium hydroxide buffer, pH9.5 at the same time. Then Hb was added at molar ratio of 3:1 with HSA and reacted for 48 h shaking at 37° C. The conjugates were detected by SDS-PAGE and bands of 83 kD and 97 kD appeared. Hb subunits can be dissociated by the detergent SDS. The molecular weights of Hb subunits are 16 kD. The molecular weight of HSA is 67 kD. Therefore, the band of 83 kD is HSA coupled with one subunit of Hb, and the band of 97 kD is HSA coupled with two cross-linked Hb subunits, which proved that Hb-HSA conjugates had been produced.

### Example 5

Two-Step Coupling of Canine Hemoglobin and HSA with Glutaraldehyde (GA)

Coupling reaction was carried out in 50 mM HEPES buffer, pH7.0. HSA was first reacted with cross-linker with a concentration of 1 mg/mL. The molar ratio of GA and HSA was 100:1. The reaction was performed at 37° C. for 1 h under shaking with total volume of 10 ml. Unreacted cross-linker may be removed by column of Sephadex G25, and the buffer was changed to 50 mM boric acid-borax buffer, pH8.5 at the same time. Then Hb was added at molar ratio of 1:3 with HSA and reacted at 37° C. for 10 h. The conjugates were detected by SDS-PAGE and a band of 83 kD appeared, which showed that Hb-HSA conjugates had been prepared.

### Example 6

### Solid-Phase Coupling of Equine Hemoglobin and HSA with Glycolaldehyde

10 mL HSA solution with concentration of 2 mg/mL is mixed with the solid media of Q Sepharose Fast Flow that have been equilibrated by 50 mM HEPES buffer, pH 6.6, followed by thorough washing till no protein was present in the eluate. Glycolaldehyde is added at molar ratio of 500:1 with HSA. The column was closed for 2 h at 10° C., and then washed extensively with the above equilibration buffer to remove excessive cross-linker, followed by elution with 0.5 M NaCl in the equilibration buffer to collect the active protein by cross-linker. The fractions may be passed through a pre-packed Sephadex G-25 gel filtration column to desalt, followed by concentration with pressure filtration, obtaining 5 mg/mL active HSA, and the pH of the solution was changed to 9.5 at the same time. Hemoglobin was then added to react for 24 h to get hemoglobin and HSA conjugates.

### Example 7

Solid-Phase Coupling of Bovine Hemoglobin and HSA with Glutaraldehyde

10 mL Hemoglobin solution with concentration of 5 mg/mL is mixed with the solid media of DEAE Sepharose Fast Flow that have been equilibrated by 50 mM PBS, pH 8.0, followed by thorough washing till no protein was present in the eluate. Glutaraldehyde is added at molar ratio of 10:1 with Hb. The column was closed for 0.5 h at 10° C., and then washed extensively with the above equilibration buffer to remove excessive cross-linker, followed by elution with 0.5 M NaCl in the equilibration buffer to collect the active protein by cross-linker. The fractions may be passed through a pre-packed Sephadex G-25 gel filtration column to desalt, followed by pressure filtration concentration, obtaining 1 mg/mL active Hb, pH 8.0. HSA with the same mole as Hb was then added to react for 8 h to get hemoglobin and HSA conjugates.

### Example 8

### Purification of the Conjugates

The Hb-HSA conjugates were purified by ultrafiltration with MWCO 300 kD and 100 kD to remove uncoupled and the conjugates whose Mws are more than 300 kD and less than 100 KD. The Mw of final product is 100-300 kD, comprising 1-3 intermolecularly or intramolecularly cross-linked hemoglobin molecules and 1-3 human serum albumin molecules.

### Example 9

### Purification of the Conjugates

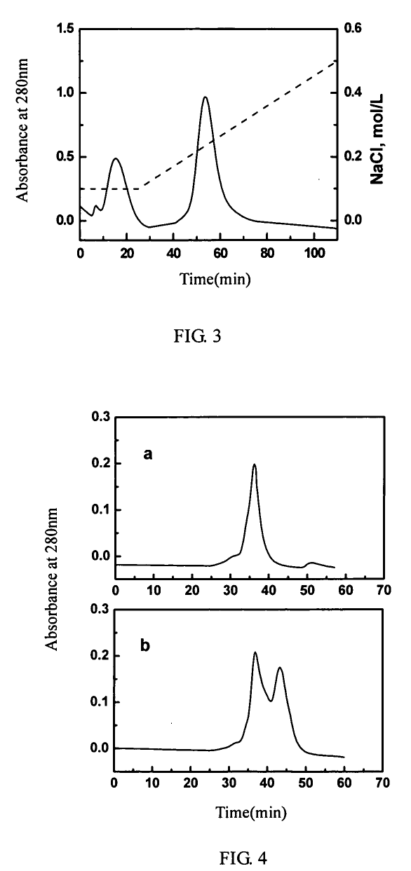

The conjugates prepared in example 6 was adjusted to pH7.0 and subjected to ion-exchange chromatography on DEAE Sepharose Fast Flow. The column was eluted with 25 ml 50 mM HEPES, containing 0.1 M NaCl, followed by elution with 55 ml, 0.1-0.5 M NaCl gradient in 50 mM HEPES buffer, pH7.0. The flow rate is 0.5 ml/min. FIG. 3 is the chromatogram illustrating the purification of the conjugates by ion exchange chromatography detected at 280 nm. The fractions containing the conjugates were pooled and loaded on the Superdex 200 gel filtration column using 50 mM HEPES buffer as eluent. The flow rate was 0.35 m/min. As shown in FIG. 4, the conjugates were eluted in the first elution peak, and the peak was then pooled and further detected by SDS-PAGE. FIG. 5 is characterization of the conjugates using SDS-PAGE. There are only two bands of 16 kD and 83 kD. Hemoglobin is a tetramer and the subunits dissociated into one subunit of 16 kD after boiling in the SDS buffer. If an Hb molecule conjugates with an HSA molecule, three subunits of Hb will dissociate, and HSA coupled with one Hb subunit shows a 83 kD band in SDS-PAGE. It can be inferred from the two bands that one-Hb-one-HSA conjugate has been prepared.

### Example 10

### Characterization of the Blood Substitute

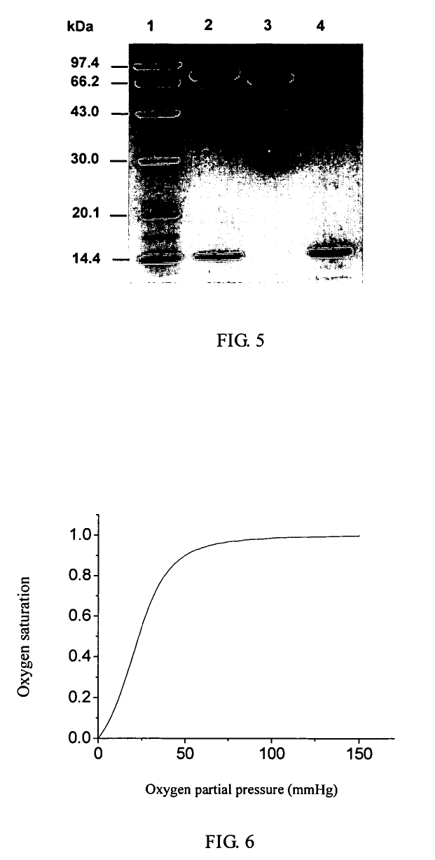

The pH value of purified Hb-HSA conjugates were adjusted to 7.4, and 2,3-diphosphoglycerate (2,3-DPG) or pyridoxal-5′-phosphate was added as covalence regulator for those hemoglobins whose ability of oxygen carrying can be effected by 2,3-DPG. The bioactivity of conjugates in example 7 was measured by Hemox™ Analyzer (TCS Scientific Corp., PA), and P₅₀ and Hill coefficient were 26.8 mmHg and 2.30 respectively. FIG. 6 is the oxygen equilibrium curves of the conjugates.

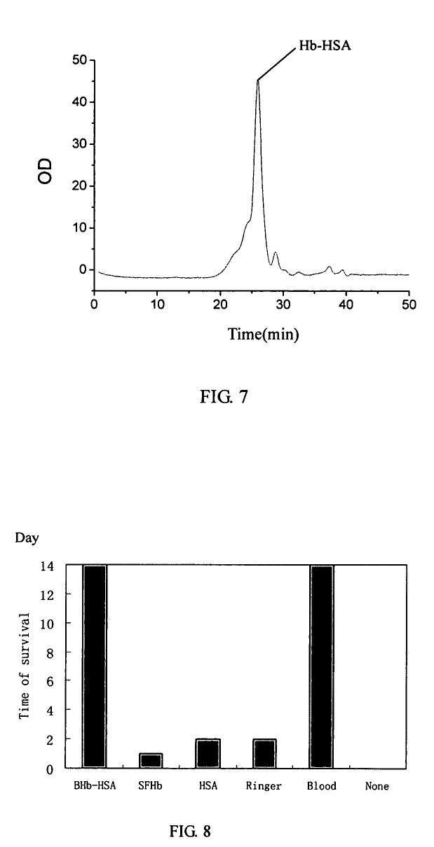

Molecular weights of Hb-HSA conjugatess were determined by the size exclusion HPLC coupled with a multi-angle laser light scattering (MALLS) detector (Wyatt Technology Co., USA). The content of each component was calculated by comparison of peak areas (see FIG. 7). The final product of Hb-HSA conjugates was simply composed of 88.5% one-Hb-one-HSA conjugate with Mw138 kD, 4.3% 2-4 protein conjugates with Mw 202 kD, 4.5% unconjugated HSA and 0.5% unconjugated Hb. The composition is simple. The colloid osmotic pressure (COP) is 21.5 mmHg, near to that of blood, 25 mmHg. Table one is the characteristics of the Hb-HSA conjugates. Many current blood substitutes have difficulties on getting a narrow Mw distribution and keeping normal COP. For example, COP of polyhemoglobin is lower than 10 mmHg, while that of PEG or POE modified hemoglobin is usually more than 70 mmHg, which may affect the balance of osmotic pressure in body.

| Components | Characteristics | |
|---|---|---|
| Hb-HSA | 7 | (g %) |
| Molecular weight | 100˜300 | kD |
| MetHb | <3% | |
| P₅₀ | 25˜30 | mmHg |
| Hill coefficient | 1.90˜2.30 | |
| pH | 7.4 | |
| Osmotic pressure | 300˜320 | mOsm |
| Virus | — | |
| Bacteria | — | |
| Pyrogen | — | |
| Lipid | — | |
| Cl⁻ | 110˜125 | mM |
| Na⁺ | 136 | mM |
| K⁺ | 3.2˜4.2 | mM |
| Ca⁺⁺ | 2.0 | mM |
| Mg⁺⁺ | 1 | mM |
| HCO₃ ⁻ | 25 | mM |
| HPO₄ ⁻ | 0.5 | mM |
| HPO₄ ²⁻ | 0.5 | mM |
| Viscose | 3.0˜8.0 | MPa · s |

| | |
|---|---|
| Impurity protein | — |
| Glutathione or others provideng thiol groups | Suitable amount |

### Example 11

### Abnormal Toxicity Test of Hb-HSA Conjugates

According to Requirements for Abnormal Toxicity Test of Biologics in Committee for Standardization of Biologics of China (2000), five ICR mices weighing 18-20 g and two guinea pigs weighing 271.1-277.7 g were given 0.5 ml and 5 ml Hb-HSA conjugates respectively by abdomen injection and fed for seven days to test the abnormal toxicity of the modified Hbs. Results showed that the animals grew actively with increasing weight in the test week, which proved that the conjugates had no abnormal toxicity.

### Example 12

Resuscitation to Hemorrhagic Shock Rats

Anesthetized Sprague-Dawley rats were fixed on ethanol disinfected operating tables. Polyethylene cannulas were filled with 0.3% heparin and placed in left femoral arteries to withdraw blood, and right femoral arteries to measure mean arterial pressure with multi-conduction physiological recorder. Hb-HSA or control solutions were infused from the left femoral vein. The 30% of total blood volume was slowly withdrawn at the speed of 0.5 mL/min, and then waited for self-complement of rats. Ten minutes later, 60% of total blood volume bleeding was finally withdrawn at the same speed. Thirty minutes later, the blood pressure decreased to 25% of the original pressure, and the same volume of Hb-HSA conjugates or controls were infused at the speed of 0.5 mL/min. We used Ringer-lactate solution equivalent to three times the volume of shed blood, stroma-free hemoglobin in Ringer-lactate solution, 5% HSA in Ringer-lactate solution, whole blood and no resuscitation fluid as controls. For each volume of bleeding, there were 6 groups of rats with 6 rats randomly assigned to each group. After the completion of infusion, rats were returned to separate cages and monitored for 14 days. The group received Hb-HSA conjugates all survived in the 14 days monitored, the same as whole blood. FIG. 8 shows the effect of one single replacement transfusion on the long term survivals in the lethal hemorrhagic shock model with 60% bleeding, which indicated that the Hb-HSA conjugates was effective in hemorrhagic shock, similar to whole blood.

### Example 13

Exchange Transfusion on Canine

Six Beagle canines were anesthetized. Blood was withdrawn from left femoral arteries by vermicular bump. Hb-HSA conjugates were infused from the right femoral vein. Mean arterial pressures were measured. Fifty percent of canine blood was exchanged and MAP was continuously recorded for 2 h. The conjugates maintained the MAP at its initial level with no pressor effect. After the completion of infusion, canines were returned to separate cages and monitored for 20 days. Two days later, the canines turned active. Within the monitored 20 days, there is no hematuria symptom and the parameters of blood were kept normal.

We claim:

1. Protein conjugates comprising hemoglobin and human serum albumin.

2. Protein conjugates according to claim 1 wherein said conjugates has a molecular weight in a range of 100-300 kD.

3. Protein conjugates according to claim 1 wherein said conjugates comprising 1-3 hemoglobin molecules and 1-3 human serum albumin molecules.

4. Protein conjugates according to claim 3 wherein said conjugates comprising 1-2 hemoglobin molecules and 1-2 human serum albumin molecules.

5. Protein conjugates according to claim 4 wherein said conjugates comprising one hemoglobin molecule and one human serum albumin molecule.

6. Protein conjugates according to claim 1 wherein the said hemoglobin is intramolecularly cross-linked.

7. A method for preparing the protein conjugates of claim 1 comprising
preparing stroma-free hemoglobin,
conjugating hemoglobin (Hb) with human serum albumin (HSA), and
purifying said Hb-HSA conjugates.

8. The method of claim 7 wherein the stroma-free hemoglobin is prepared by membrane filtration and ion exchange chromatography, comprising the steps of:
Processing through microfiltration membranes with mean pore size from 0.22 μm to 0.65 μm, followed by ultrafiltration with membranes of molecular weight cut-off from 10 kD to 30 kD;
The pretreated hemoglobin solution further purified by anion exchange chromatography in flow-through mode at 4-10° C., with 10-50 mM buffer, pH 6.6-8.5, using 0.25-10% polyethylene glycol (PEG) 400-4000 as an escort.

9. The method of claim 7 wherein Hb and HSA conjugation methods is either one-step or two-step coupling, wherein in two step coupling, the cross-linker reacts first with one protein either in solution or on solid medium, then reacts with another protein in solution.

10. The method of claim 7 wherein the purification of Hb-HSA conjugates comprising either one, two or three of the methods selected from ion exchange chromatography, ultrafiltration and gel filtration chromatography.

11. A method for using the hemoglobin conjugates of claim 1 wherein said conjugates are used as blood substitutes.
